(19) United States
(12) Patent Application Publication
(10) Pub. No.: US 2024/0034986 A1
(43) Pub. Date: Feb. 1, 2024

(54) SUPPLEMENT COMPOSITION FOR CELL CULTURE

(71) Applicant: CLEAR MEAT PRIVATE LIMITED, Noida (IN)

(72) Inventors: Pawan K DHAR, Greater Noida (IN); Siddharth MANVATI, Noida (IN)

(21) Appl. No.: 18/257,499

(22) PCT Filed: Dec. 13, 2021
(86) PCT No.: PCT/IB21/61622

§ 371 Date: Jun. 14, 2023

(30) Foreign Application Priority Data
Dec. 14, 2020 (IN) 202011054395

(51) Int. Cl. C12N 5/00

(52) U.S. Cl. CPC C12N 5/0018; C12N 2500/10; C12N 2500/32; C12N 2500/34; C12N 2500/36; C12N 2500/76; C12N 2500/05

(57) ABSTRACT

The present invention provides a supplement composition and method of preparation thereof for cell culture. The supplement composition comprises natural substances without using any animal based serum. The present invention provides slaughter free, effective and affordable supplement for growth of animal cells that eliminates or minimizes dependency on the fetal bovine serum.

### FIELD OF INVENTION

The present invention relates to a supplement composition for cell culture and method of preparation thereof. Particularly, the present invention relates to the supplement composition comprises natural substances and provides significant growth rate of cells. More particularly, the present invention relates to a supplement composition that supports cell growth without adding any animal-derived substance and basal media.

### BACKGROUND OF THE INVENTION

Due to a drastic development in the field of life sciences and biopharmaceutical industry, a huge demand for the cell-culture media and its supplement has been raised. The most beneficial supplement used for the cell-culture media is derived from animals; such as fetal bovine serum (FBS). The FBS contains attachment and growth factors, as well as the nutritional and physiochemical components essential for cell maintenance and growth. Despite having the beneficial effects, it has a number of limitations and drawbacks.

It is difficult to maintain the quality and consistency of FBS as it varies from batch to batch, thereby posing major challenges in scientific research and cell culture-based companies. Further, processing of FBS is difficult as it is an animal based product and readily gets contaminated by bacteria, viruses, fungi and most commonly by Mycoplasma. Also, ethical concern is a major issue associated with FBS as procurement of bovine serum demands the slaughtering of numerous calves and contributes to environmental damage through gas emissions. Concerns regarding FBS safety and inadequacies in cell culture have grown in recent years, emphasizing the need for FBS substitutes. Nonetheless, despite significant efforts in the production of different FBS-free media, complete research and assessment of such media is still required.

Several researchers have already been done for providing the alternatives to growth media such as chemically defined medium replacements, tissue extracts (e.g., pituitary extracts), ocular fluid, bovine milk fractions or bovine colostrum, plant extracts (vegetal serum), and platelet lysates have all been commercially accessible or are currently being investigated.

Keeping in view the problems associated with the present state of art, there is an urgent need for slaughter free, effective and sustainable supplement for cell culture having growth supporting characteristics for animal cell lines, and provides enhanced effects as compared to the animal-based serum.

### OBJECTIVE OF THE INVENTION

The primary objective of the present invention is to provide a slaughter free, effective and affordable supplement for growth of animal cells that eliminates or minimizes dependency on the fetal bovine serum.

Another objective of the present invention is to increase the growth rate of the cells without using any animal-derived supplement and other basal media.

Yet another objective of the present invention is to provide a supplement composition comprising only natural substances.

Yet another objective of the present invention is to provide cell viability in the range of 90-99% after 72 hours with a significant increase in the number of healthy cells.

Yet another objective of the present invention is to maintain the morphology of cells and minimizing the number of dead cells.

Yet another objective of the present invention is to provide a supplement composition useful for all types of cell lines.

Other objectives and advantages of the present invention will become apparent from the following description taken in connection with the accompanying drawings, wherein, by way of illustration and example, the aspects of the present invention are disclosed.

### SUMMARY OF THE INVENTION

The present invention relates to a supplement composition and method of preparation thereof for cell culture media. A supplement composition for cell culture comprising natural substances that supports cell growth without any further addition of animal based serum. The composition particularly, comprises the following components: inorganic salts in a range of 0-40 wt %; amino acids in a range of 0-10 wt %; vitamins in a range of 0-1 wt %; lipids in a range of 0-10 wt %; pigments in a range of 0-1 wt %; trace elements in a range of 0-3 wt %, other compounds in a range of 0-35 wt % and mixture thereof. The composition provides the highest cell viability and proliferation rate along with the maintained morphology of cell.

### BRIEF DESCRIPTION OF FIGURES

FIG. 1 illustrates the flow chart for preparing the supplement composition.

FIG. 2 illustrates cell proliferation rate of Clear X9 induced cell growth of HEK293 cells compared with DMEM with 10% FBS media containing HEK293 cells at 72 hours.

FIG. 3 illustrates cell proliferation rate of 100% Clear X9 induced cell growth of MDA-MB-231, MCF-7 and HEK293 cells Vs DMEM with 10% FBS containing cells at 24, 48 and 72 hours.

FIGS. 4A and 4B illustrate cell viability of 100% Clear X9 induced cell growth of MDA-MB-231, MCF-7 and HEK293 cells at 72 hours.

FIG. 5 illustrates cell morphology of Clear X9 induced cell growth of HEK293 cells at 72 hours.

FIG. 6 illustrates cell morphology of 100% Clear X9 induced cell growth of MDA-MB-231, MCF-7 and HEK293 cells at 72 hours.

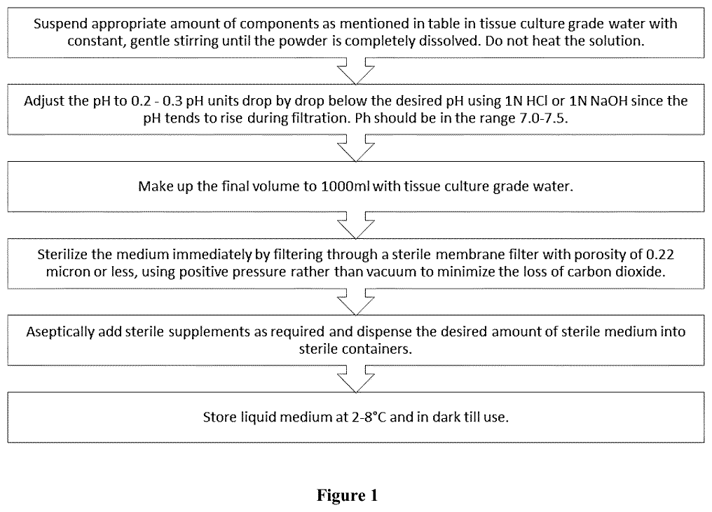

### DETAILED DESCRIPTION OF THE INVENTION

The following detailed description and embodiments set forth herein below are merely exemplary out of the wide variety and arrangement of instructions which can be employed with the present invention. The present invention may be embodied in other specific forms without departing from the spirit or essential characteristics thereof. All the features disclosed in this specification may be replaced by similar other or alternative features performing similar or same or equivalent purposes. Thus, unless expressly stated otherwise, they all are within the scope of the present invention.

Accordingly, those of ordinary skill in the art will recognize that various changes and modifications of the embodiments described herein can be made without departing from the scope of the invention. In addition, descriptions of well-known functions and constructions are omitted for clarity and conciseness.

The terms and words used in the following description and claims are not limited to the bibliographical meanings but are merely used to enable a clear and consistent understanding of the invention. Accordingly, it should be apparent to those skilled in the art that the following description of exemplary embodiments of the present invention are provided for illustration purpose only and not for the purpose of limiting the invention.

It is to be understood that the singular forms “a”, “an” and “the” include plural referents unless the context clearly dictates otherwise.

It should be emphasized that the term “comprises/comprising” when used in this specification is taken to specify the presence of stated features, integers, steps or components but does not preclude the presence or addition of one or more other features, integers, steps, components or groups thereof.

It should be noted that the term “Clear X9” used herein the description refers to the novel supplement composition for cell culture media.

Further, it to be noted that the term “tissue culture grade water” used herein the description refers to the highly purified water.

Accordingly, the present invention provides a supplement composition comprising the natural substances which provides the enhancements in cell growth including increased growth rates, growth to high cell densities.

The supplement composition for cell culture comprises inorganic salts, amino acids, vitamins, lipids, pigments, trace elements and other additive compounds. The supplement composition works as a complete growth media and may not need any basal media to provide the significant growth rate of cells.

In an embodiment of the invention, the supplement composition comprises one or more of the components:
a. at least one inorganic salt [0-40 wt %];
b. at least one amino acid[0-10 wt %];
c. at least one vitamin [0-1 wt %];
d. at least one lipid [0-10 wt %];
e. at least one pigment [0-1 wt %];
f. at least one trace element [0-3 wt %]; and
g. at least one additive/excipient 0-35 wt %; or mixtures thereof.

The cell culture supplement comprising one or more inorganic salts, selected from, but not limited to Calcium chloride (CaCl₂), Ferric nitrate (Fe(NO₃)₃-9H₂O), Potassium chloride (KCl), Magnesium sulfate (MgSO₄), Sodium chloride (NaCl), Sodium bicarbonate (NaHCO₃), Sodium phosphatemonohydrate (NaH₂PO₄—H₂O). The concentration of inorganic salts used in the composition is between 0-25 g/L.

The cell culture supplement comprising one or more amino acids, selected from, but not limited to L-Arginine hydrochloride, L-Cystine, L-Glutamine, Glycine, L-Histidine-HCl—H2O, L-Isoleucine, L-Leucine, L-Lysine hydrochloride, L-Methionine, L-Phenylalanine, L-Serine, L-Threonine, L-Tryptophan, L-Tyrosine and L-Valine. The concentration of amino acids used in the composition is between 0-3 g/L.

The cell culture supplement comprising one or more vitamins, selected from, but not limited to Biotin, D-Calcium pantothenate, Choline chloride, Folic acid, i-Inositol, Niacinamide, Pyridoxal hydrochloride, Pyridoxine hydrochloride, Riboflavin, Thiamine hydrochloride and Vitamin B12. The concentration of vitamins used in the composition is between 0-2 g/L.

The cell culture supplement comprising one or more lipids, selected from, but not limited to the lipids are selected from group consisting of oleic acid, palmitic acid, alpha-linoleic acid, stearic acid, heptadecanoic acid, and doconexent. The concentration of lipids used in the composition is between 0-10 g/L.

The cell culture supplement comprising one or more pigments, selected from, but not limited to leutine, beta-carotinal, and beta-cryptoxanthin. The concentration of pigments used in the composition is between 0-0.5 g/L.

The cell culture supplement comprising one or more trace elements, selected from, but not limited to potassium, sodium, calcium, magnesium, phosphorus, iron, zinc, copper, nickel and chromium. The concentration of trace elements used in the composition is between 0-1 g/L.

The cell culture supplement comprising one or more other compounds/additives/excipents, selected from, but not limited to saccharide, toxins, growth factors, sodium pyruvate, pH indicators such as phenol red, 2-phenyl-2-butenal and buffers such as HEPES. The concentration of other compounds used in the composition is between 0-25 g/L.

The saccharide is selected from sucrose, mannose, glucose, galactose, fructose and maltose in combination thereof.

In a preferred embodiment, D-glucose acts an energy source to the cell culture media.

In the preferable embodiment, toxin is endotoxin having the concentration ranging between 0-0.1 g/L.

In the preferable embodiment, the growth factor is Chlorella growth factor having the concentration ranging between 0-0.0001 g/L.

A broad range of constituents in the present invention is tabulated below:

TABLE 1

| Constituents | Parts by Weight |
|---|---|
| Inorganic Salts | 0%-40% |
| Amino Acids | 0%-10% |
| Vitamins | 0%-01% |
| Lipids | 0%-10% |
| Pigments | 0%-01% |
| Trace Elements | 0%-03% |
| Additives/excipients | 0%-35% |

In an exemplary embodiment, the present invention relates to a method for preparing the composition comprising the steps of:
a. suspending components in water with continuous stirring to obtain a completely dissolved solution;
b. adjusting the pH of the solution of step (a) in the range of 7.2 to 7.5 by adding 1N acid or base drop by drop followed by adding water to adjust volume of the solution;
c. sterilizing the solution as obtained in step (b) by filtering through a sterile membrane filter under positive pressure to obtain a filtered solution;
d. aseptically adding growth factors and vitamins into the filtered solution obtained in step (c) to obtain the supplement composition; and
e. storing the supplement composition obtained from step (d) in a sterile container at a temperature of 2-8° C. in dark.

The components used in step (a) are inorganic salts, amino acids, lipids, pigments, trace elements and additives/excipients.

The acid/base used in step (b) is selected from, but not limited to, 1N HCl and 1N NaOH.

In an embodiment, the pH of the solution is adjusted within the range of 7.0-7.5 by adjusting the pH to 0.2-0.3 pH units dropwise below the desired pH since pH tends to rise during filtration.

The porosity of sterile membrane is in the range of 0.10 to 0.22 micron and the filtration is performed at the positive pressure rather than vacuum to minimize the loss of carbon dioxide.

The supplement composition is stored at a temperature 2-8° C. in dark environment.

The supplement composition supports the growth of animal tissue cells selected from group consisting of pluripotent stem cells, embryonic stem cells, bone marrow stromal cells, hematopoietic progenitor cells, lymphoid stem cells, myeloid stein cells, T cells, B cells, macrophages, hepatic cells, pancreatic cells, carcinoma cells and cell lines.

The cell lines are selected from, but not limited to, human breast cancer cell and human embryonic kidney cells.

In an exemplary embodiment, cell lines are selected from, but not limited to, MDA-MB-231, MCF-7 and HEK-239.

In an exemplary embodiment of the present invention, the supplement composition for cell culture shows the highest cell viability and proliferation rates along with the maintaining the cell morphology. MTT assay is used herein, measures the cellular metabolic activity as an indicator of cell viability, proliferation and cytotoxicity. The term used “Cell viability” defines as to measure of the proportion of live, healthy cells within a population and are determined with the help of Cell viability assays (Typan Blue assay) conducted on optimize conditions. The obtained data can be calculated by following formula:

Percentage of viable cells: No. of viable cells (unstained)/Total no. of the cells×100

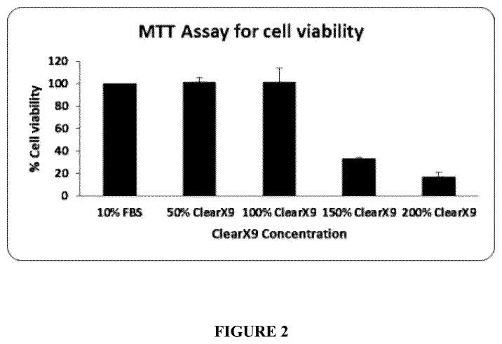

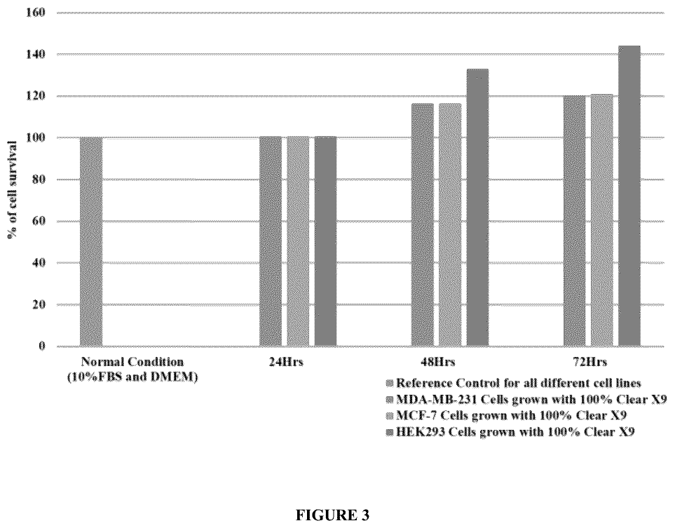

In an embodiment, the comparative data of DMEM basal media with FBS (Fetal Bovine Serum) and supplement composition (Clear X9) are represented in FIG. 2-6. FIG. 2 provides the cell viability comparative data of DMEM basal Media with 10% FBS and Clear X9 having concentration in the range of 50-200%. FIG. 3 provides the cell survival data from a normal condition to a period of 72 hours.

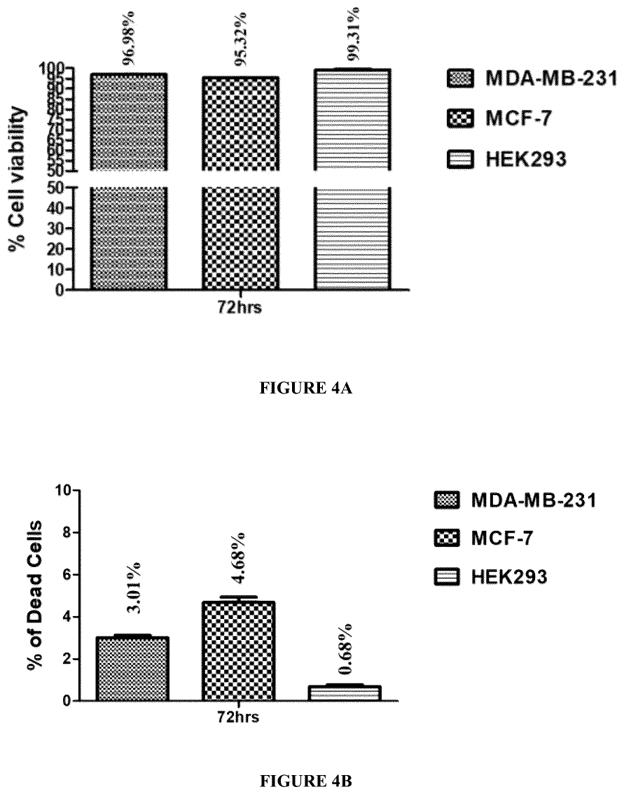

The pilot studies conducted have demonstrated viability of cells in the range of 90%-99% after 72 hours with a significant increase in the number of healthy cells. (FIG. 4A-4B).

As per an exemplary embodiment, the following tabulates the composition as per present invention:

TABLE 2

| | |
|---|---|
| Components of Clear X9 | Approx. final conc (g/L) |

Inorganic Salts

| | |
|---|---|
| Calcium chloride (CaCl₂) | 0.0-0.40 |
| Iodine | 0.0-0.01 |
| Ferric nitrate (Fe(NO₃)₃—9H₂O) | 0.0-0.40 |
| Potassium chloride (KCl) | 0.0-1.00 |
| Magnesium sulfate (MgSO₄) | 0.0-0.40 |
| Sodium chloride (NaCl) | 3.0-10.00 |
| Sodium bicarbonate (NaHCO₃) | 3.0-10.00 |
| Sodium phosphate, monohydrate | 0.0-1.00 |
| (NaH₂PO₄—H₂O) | |

Amino Acids

| | |
|---|---|
| L-Arginine hydrochloride | 0.0-1.00 |
| L-Cystine | 0.0-1.00 |
| L-Glutamine | 0.0-1.00 |
| Glycine | 0.0-0.40 |
| L-Histidine-HCl—H₂O | 0.0-0.40 |
| L-Isoleucine | 0.0-1.00 |
| L-Leucine | 0.0-1.00 |
| L-Lysine hydrochloride | 3.0-10.00 |
| L-Methionine | 0.0-1.00 |
| L-Phenylalanine | 0.0-1.00 |
| L-Serine | 0.0-1.00 |
| L-Threonine | 0.0-1.00 |
| L-Tryptophan | 0.0-1.00 |
| L-Tyrosine | 0.0-1.00 |
| L-Valine | 0.0-1.00 |
| Epsilon-Polylysine | 0.0-1.00 |
| erythro-Isoleucine | 0.0-1.00 |
| Asparagine | 0.0-1.00 |
| Glutamic acid | 0.0-1.00 |
| Arpartic acid | 0.0-1.00 |
| Proline | 0.0-1.00 |

Vitamins

| | |
|---|---|
| Ratinol | 0.0-1.00 |
| Ergocalciferol | 0.0-1.00 |
| D-Calcium pantothenate | 0.0-1.00 |
| Choline chloride | 0.0-1.00 |
| Folic acid | 0.0-1.00 |
| i-Inositol | 0.0-1.00 |
| Niacinamide | 0.0-1.00 |
| Pyridoxal hydrochloride | 0.0-1.00 |
| Pyridoxine hydrochloride | 0.0-1.00 |
| Riboflavin | 0.0-1.00 |
| Thiamine hydrochloride | 0.0-1.00 |
| Vitamin B12 | 0.0-1.00 |
| Nicotinic acid | 0.0-0.001 |
| alpha-Tocopherol | 0.0-0.001 |

Lipids

| | |
|---|---|
| Oleic acid | 0.0-3.0 |
| Palmitic acid | 0.0-3.0 |
| Alpha-Linoleic acid | 0.0-1.0 |
| Stearic acid | 0.0-1.0 |
| Heptadecanoic acid | 0.0-0.01 |
| Doconexent | 0.0-0.10 |

Pigments

| | |
|---|---|
| Leutine | 0.0-0.10 |
| Beta-Carotinal | 0.0-0.10 |
| Beta-Cryptoxanthin | 0.0-0.01 |

Trace Elements

| | |
|---|---|
| Potassium | 0.0-0.10 |
| Sodium | 0.0-0.10 |
| Calcium | 0.0-0.10 |
| Magnesium | 0.0-0.01 |
| Phosphorus | 0.0-0.01 |
| Iron | 0.0-0.10 |
| Zinc | 0.0-0.10 |
| Copper | 0.0-0.01 |
| Nickel | 0.0-0.10 |
| Chromium | 0.0-0.10 |

Additives/Excipients

| | |
|---|---|
| D-Glucose | 3.0-10.00 |
| 2-Phenyl-2-butenal | 0.0-1.50 |
| Sucrose | 0.0-0.10 |
| Chlorella Growth Factors | 0.0-0.0001 |
| Endotoxins | 0.0-0.01 |
| Phenol red | 0.0-1.00 |
| Sodium Pyruvate | 0.0-1.00 |
| HEPES (4-(2-hydroxyethyl)-1- | 3.0-10.00 |
| piperazineethanesulfonic acid) | |

### EXAMPLES

The following examples are given by way of illustration of the working of the invention in actual practice and should not be constructed to limit the scope of the present invention in any way.

### Example 1

Suspend appropriate amount of components as mentioned in table no. 3 except growth factors and vitamins in tissue culture grade water with constant, gentle stirring until the powder is completely dissolved. Do not heat the solution.
Adjust the pH to 0.2-0.3 pH units drop by drop below the desired pH using 1N HCl or 1N NaOH since the pH tends to rise during filtration. pH should be in the range 7.0-7.5.
Make up the final volume to 1000 ml with tissue culture grade water.
Sterilize the medium immediately by filtering through a sterile membrane filter with porosity of 0.22 micron or less, using positive pressure rather than vacuum to minimize the loss of carbon dioxide.
Aseptically add Growth factors and vitamins as required and dispense the desired amount of sterile medium into sterile containers.
Store liquid medium at 2-8° C. and in dark till use.
   Table 3 represents the amount of the components added during the preparation of composition according to an embodiment of the invention.

| | |
|---|---|
| COMPONENT | g/L |

Inorganic Salts

| | |
|---|---|
| Calcium chloride (CaCl₂) | 0.2000 |
| Iodine | 0.0050 |
| Ferric nitrate (Fe(NO₃)₃—9H₂O) | 0.0005 |
| Magnesium sulfate (MgSO₄) | 0.0898 |
| Potassium chloride (KCl) | 0.5000 |
| Sodium bicarbonate (NaHCO₃) | 5.1010 |
| Sodium chloride (NaCl) | 7.0010 |
| Sodium phosphate, monohydrate | 0.0990 |
| (NaH₂PO₄—H₂O) | |

Amino Acids

| | |
|---|---|
| L-Arginine hydrochloride | 0.9100 |
| L-Cystine | 0.0737 |
| L-Glutamine | 0.7010 |
| Glycine | 0.0400 |
| L-Histidine-HCl—H2O | 0.0420 |
| L-Isoleucine | 0.1110 |
| L-Leucine | 0.1110 |
| L-Lysine hydrochloride | 0.1500 |
| L-Methionine | 0.0500 |
| L-Phenylalanine | 0.0606 |
| L-Serine | 0.0402 |
| L-Threonine | 0.1050 |
| L-Tryptophan | 0.0190 |
| L-Tyrosine | 0.1066 |
| L-Valine | 0.0940 |
| Epsilon-Polylysine | 0.0402 |
| erythro-Isoleucine | 0.0412 |
| L-Asparagine | 0.0422 |
| Glutamic acid | 0.0400 |
| Aspartic acid | 0.0402 |
| L-Proline | 0.0492 |

Vitamins

| | |
|---|---|
| Retinol | 0.0050 |
| Ergocalciferol | 0.0050 |
| D-Calcium pantothenate | 0.0072 |
| Choline chloride | 0.0050 |
| Folic acid | 0.0040 |
| I-Inositol | 0.0050 |
| Niacinamide | 0.0050 |
| Pyridoxal hydrochloride | 0.0005 |
| Pyridoxine hydrochloride | 0.0050 |
| Riboflavin | 0.0005 |
| Thiamine hydrochloride | 0.0050 |
| Vitamin B12 | 0.0050 |
| Nicotinic acid | 0.0050 |
| alpha-Tocopherol | 0.0050 |

Lipids

| | |
|---|---|
| Oleic acid | 2.478 |
| Palmitic acid | 1.275 |
| Alpha-Linoleic acid | 0.5868 |
| Stearic acid | 0.4051 |
| Doconexent | 0.0635 |

Pigments

| | |
|---|---|
| Leutine | 0.03975 |
| Beta-Cryptoxanthin | 0.04521 |
| Beta-Carotinal | 0.02912 |

Trace Elements

| | |
|---|---|
| Potassium | 0.0700 |
| Sodium | 0.0530 |
| Calcium | 0.0230 |
| Magnesium | 0.0070 |
| Phosphorus | 0.0065 |
| Iron | 0.0012 |
| Zinc | 0.0007 |
| Copper | 0.0005 |
| Nickel | 0.0002 |
| Chromium | 0.0003 |

Additives/Excipients

| | |
|---|---|
| D-Glucose | 5.1001 |
| 2-Phenyl-2-butenal | 0.0014 |
| Sucrose | 0.0072 |
| Chlorella Growth Factors | 0.0000005 |
| Endotoxins | 0.0111 |
| Phenol red | 0.0210 |
| Sodium Pyruvate | 0.1211 |
| HEPES (4-(2-hydroxyethyl)-1- | 6.0102 |
| piperazineethanesulfonic acid) | |

### Materials

### Cell Line and Reagents:

MDA-MB-231, MCF-7 and HEK293 cell lines were obtained from American Type Culture Collection (Manassas, VA) and grown in DMEM medium (Invitrogen) supplemented with 10% heat-inactivated fetal bovine serum (Invitrogen) or Clear X9 complete growth media at different percentage (50, 100, 150 and 200%) at 5% CO₂, 37° C. under a humidified chamber. Trypan blue dye (C.I. 23850) and MTT or “[3-(4,5-Dimethylthiazol-2-Yl)-2,5-Diphenyltetrazolitim Bromide]” dye (M2128) was obtained from Sigma Aldrich. Triple distilled water prepared by “Millipore” was used in making the reagents. Other routine chemicals were obtained in their commercially available highest purity grade.

### Example 2

### Cell Viability Assay

Cells (7×105/ml) were plated in 2 ml Clear X9 complete growth media (50% to 200%) at 5% CO₂, 37° C. under a humidified chamber for 72 hours. At the end of the stipulated time interval, cells were harvested, washed twice with 1×PBS and collected in separate tubes. For viability assay, 100 μl volume of the homogeneous cell suspension was transferred to micro-centrifuge tube and 10111 of 0.5% trypan blue dye was added. The suspension was mixed well and left for 5-10 minutes at room temperature. 12 μl of stained cell suspension was loaded on each chamber of the hemocytometer. Unstained cells were counted as live and blue stained cells as dead cell in an inverted microscope and the procedure was repeated for each set of all samples. Each sample was counted in triplicate and total cell number of live & dead cells was determined.

Percentage of viable cells: No. of viable cells (unstained)/Total no. of the cells×100.

### Example 3

### MTT Assay

Effect of either the DMEM media supplemented with 10% Fetal Bovine Serum (FBS) or 50-200% Clear X9 complete growth media on MDA-MB-231, MCF-7 and HEK293 cells proliferation rate were measured using MTT assay. Briefly, all cells (5×104/ml) were plated (approximately 10,000/well) in triplicate in 96 well plates. The cells were incubated in the presence of effect of either the DMEM media supplemented with 10% Fetal Bovine Serum (FBS) or 50-100% Clear X9 complete growth media in a final volume of 200 μl for different time lengths (24, 48 and 72 hrs) at 37° C. in a 5% CO₂ humidified chamber. Cells grown with 10% FBS served as a control. At the end of each time point, 20 μl of MTT solution (5 mg/ml in 1×PBS) added to each well and incubated for another 5 hours. After discarding the supernatant at the end of 5 hours, the resultant formazan crystals produced were dissolved in 200 μM of Dimethyl sulfoxide (DMSO). The absorbance value (A) measured at 570 nm by a microplate ELISA reader.

### Example 4

### Cell Morphology Study

Cells (7×105/ml) were plated in 2 ml DMEM medium with 10% Fetal Bovine Serum (FBS) or Clear X9 complete growth media (50 to 200%) in 35 mm2 culture dishes for 72 hours at 5% CO₂, 37° C. under a humidified chamber. Bright field microscopy was used for studying the morphology in response to the presence of DMEM complete medium or Clear X9 complete media. All images were captured at 5× magnification using bright field microscope (NIKON ECLIPSE Ti-S, Tokyo, Japan).

Statistical Analysis:

Statistical analysis was carried out using Graph Pad Prism software. Experimental data are expressed as means±S.D.

### Example 5

### Results and Analysis

100% Clear X9 induces the high rate of cell proliferation of MDA-MB-231, MCF-7 and HEK293 cells than 10% FBS in DMEM containing cells:

Cell viability was evaluated on breast cancer cell lines (MDA-MB-231 and MCF-7 cells), and human embryonic kidney 293 (HEK-293) cells using Trypan blue exclusion and MTT assay. Proliferation rate performance of breast cancer cells and human embryonic kidney 293 cells in Clear X9 complete growth media (50 to 200%) and DMEM with 10% FBS at 24, 48 and 72 hours.

HEK293 cells were grown in standard DMEM media with 10% FBS or Clear X9 complete growth media (50 to 200%) for 72 hours and evaluate the cell viability by MTT assay (FIG. 2).

FIG. 2 Clear X9 induced cell growth of HEK293 cells than DMEM with 10% FBS media containing HEK293 cells at 72 hours. Cell proliferation rate was done by MTT assay on HEK293 cells. Briefly, cells (5×104 cells/ml) were plated in 96 well plates and grown with either DMEM with 10% FBS or Clear X9 (50 to 200%) for 72 hours. After end of the stipulated time point MTT dye was added and incubated for 5 hours in a CO2 incubator. The formed crystals were dissolved in DMSO and OD was taken at 570 nM. The data shown are the mean from three parallel experiments, and each experiment was done in triplicate.

Similarly, MDA-MB-231, MCF-7 and HEK293 cells were grown in DMEM media with 10% FBS or 100% Clear X9 complete growth media for 24, 48 and 72 hours (FIG. 3) and check cell proliferation rate by MTT assay.

FIG. 3. 100% Clear X9 induced cell growth of MDA-MB-231, MCF-7 and HEK293 cells Vs DMEM with 10% FBS containing cells at 24, 48 and 72 hours. Cell proliferation rate was done by MTT assay on MDA-MB-231, MCF-7 and HEK293 cells. Briefly, cells (5×104 cells/ml) were plated in 96 well plates and grown with either DMEM with 10% FBS or 100% Clear X9 complete growth media for 24, 48 and 72 hours time period. After end of the stipulated time interval MTT dye was added and incubated for 5 hours in a CO₂ incubator. The formed crystals were dissolved in DMSO and OD was taken at 570 nM. The data shown are the mean from three parallel experiments, and each experiment was done in triplicate. Likewise, MDA-MB-231, MCF-7 and HEK 293 cells were grown in 100% Clear X9 complete growth media with for 72 hours and evaluate the cell viability by Trypan blue exclusion assay (FIGS. 4A and 4B).

FIGS. 4A and B. 100% Clear X9 induced cell growth of MDA-MB-231, MCF-7 and HEK293 cells at 72 hours. Effect of 100% Clear X9 on cell viability of MDA-MB-231, MCF-7 and HEK293 cells for 72 hours by Trypan Blue Assay. Data are represented as the mean±SD of three independent experiments. All results suggested that, proliferation rate of all cell types was increased in the presence of 100% Clear X9 than DMEM with 10% FBS at different time interval.

100% Clear X9 shown highly cell growth rate of breast cancer and human embryonic kidney 293 cells than DMEM with 10% fetal bovine serum containing cells:

Cell morphology in presence of either Clear X9 complete growth media or DMEM with 10% FBS was examined under bright field microscope. At 72 hours, the cell counts significantly increased in the presence of Clear X9 but unfortunately at the higher percentage of Clear X9 above to 100%, decreased growth rate was observed. Maximum growth rate (cell numbers) was observed in 100% Clear X9 set as compare to the DMEM with 10% FBS containing cells (FIG. 5).

FIG. 5. Clear X9 induced cell growth of HEK293 cells at 72 hours. Cells were plated at 7×105 cells/ml in 35 mm culture dish and grown with either Clear X9 (50, 100, 150 and 200%) and DMEM with 10% FBS, cell morphology was visualized after 72 hours using bright field microscope (Images were captured by Nikon inverted microscope at 5× magnification).

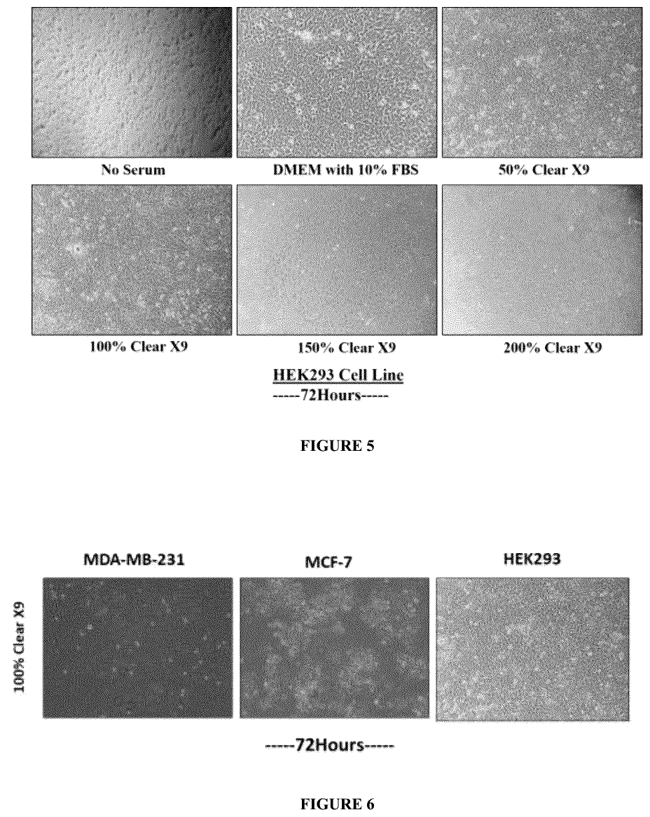

Similarly, breast cancer cells (MDA-MB-231 and MCF-7) and human embryonic kidney 293 cells were grown in 100% Clear X9 complete growth media for 72 hours (FIG. 6).

FIG. 6. 100% Clear X9 induced cell growth of MDA-MB-231, MCF-7 and HEK293 cells at 72 hours. Cells were plated at 7×105 cells/ml in 35 mm culture dish and grown with 100% Clear X9 complete growth media and cell morphology was visualized after 72 hours using bright field microscope (Images were captured by Nikon inverted microscope at 5× magnification).

After end of the stipulated time interval, breast cancer cells were growing fast but maximum growth was observed in HEK-293 (FIG. 6).

### DISCUSSION

The cell viability data clearly suggest that, 100% Clear X9 complete growth media containing cells are showing faster growth rate than DMEM with 10% FBS containing cells with significantly increasing cell numbers. Cellular morphology is often used to characterize cellular health. Clear X9 significantly increase the breast cancer and human embryonic kidney 293 cells growth at 72 hours and no morphological change was observed as compare to the DMEM with 10% FBS containing cells.

### CONCLUSION

Clear X9 containing cells are showing faster growth rate than DMEM with 10% FBS containing cells and no significantly morphological change was observed in both culture condition.

While this invention has been described in connection with what is presently considered to be the most practical and preferred embodiment, it is to be understood that the invention is not limited to the disclosed embodiments, but, on the contrary, is intended to cover various modifications and equivalent arrangements included within the scope of claims that will follow in the complete specification.

1-20. (canceled)

21. A supplement composition for cell culture comprising:
a. at least an inorganic salt in a range of 0.0005-40 wt %;
b. at least an amino acid in a range of 0.01-10 wt %;
c. at least a vitamin in a range of 0.0005-1 wt %;
d. at least a lipid in a range of 0.06-10 wt %;
e. at least a pigment in a range of 0.02-1 wt %;
f. at least a trace element in a range of 0.0002-3 wt %
g. at least an additive/excipient selected from saccharide, toxins, growth factors, sodium pyruvate, phenol red, 2-phenyl-2-butenal and HEPES in a range of 0.01-35 wt %.

22. The composition as claimed in claim 21, wherein the supplement composition comprises:

| | |
|---|---|
| Inorganic Salts | 1. Calcium chloride (CaCl₂) -0.2000 g/L |
| | 2. Iodine -0.0050 g/L |
| | 3. Ferric nitrate (Fe(NO₃)₃—9H₂O) -0.0005 g/L |
| | 4. Magnesium sulfate (MgSO₄) -0.0898 g/L |
| | 5. Potassium chloride (KCl) -0.5000 g/L |
| | 6. Sodium bicarbonate (NaHCO₃) -5.1010 g/L |
| | 7. Sodium chloride (NaCl) -7.0010 g/L |
| | 8. Sodium phosphate, monohydrate (NaH₂PO₄—H₂O) - |
| | 0.0990 g/L |
| Amino Acids | 1. L-Arginine hydrochloride- 0.9100 g/L |
| | 2. L-Cystine- 0.0737 g/L |
| | 3. L-Glutamine- 0.7010 g/L |
| | 4. Glycine- 0.0400 g/L |
| | 5. L-Histidine-HCl—H2O- 0.0420 g/L |
| | 6. L-Isoleucine- 0.1110 g/L |
| | 7. L-Leucine- 0.1110 g/L |
| | 8. L-Lysine hydrochloride- 0.1500 g/L |
| | 9. L-Methionine- 0.0500 g/L |
| | 10. L-Phenylalanine- 0.0606 g/L |
| | 11. L-Serine- 0.0402 g/L |
| | 12. L-Threonine- 0.1050 g/L |
| | 13. L-Tryptophan- 0.0190 g/L |
| | 14. L-Tyrosine- 0.1066 g/L |
| | 15. L-Valine- 0.0940 g/L |
| | 16. Epsilon-Polylysine- 0.0402 g/L |
| | 17. erythro-Isoleucine- 0.0412 g/L |
| | 18. L-Asparagine- 0.0422 g/L |
| | 19. Glutamic acid- 0.0400 g/L |
| | 20. Aspartic acid- 0.0402 g/L |
| | 21. L-Proline- 0.0492 g/L |
| Vitamins | 1. Retinol- 0.0050 g/L |
| | 2. Ergocalciferol- 0.0050 g/L |
| | 3. D-Calcium pantothenate- 0.0072 g/L |
| | 4. Choline chloride- 0.0050 g/L |
| | 5. Folic acid- 0.0040 g/L |
| | 6. I-Inositol- 0.0050 g/L |
| | 7. Niacinamide- 0.0050 g/L |
| | 8. Pyridoxal hydrochloride- 0.0005 g/L |
| | 9. Pyridoxine hydrochloride- 0.0050 g/L |
| | 10. Riboflavin- 0.0005 g/L |
| | 11. Thiamine hydrochloride- 0.0050 g/L |
| | 12. Vitamin B12- 0.0050 g/L |
| | 13. Nicotinic acid- 0.0050 g/L |
| | 14. alpha-Tocopherol- 0.0050 g/L |
| Lipids | 1. Oleic acid- 2.478 g/L |
| | 2. Palmitic acid- 1.275 g/L |
| | 3. Alpha-Linoleic acid- 0.5868 g/L |
| | 4. Stearic acid- 0.4051 g/L |
| | 5. Doconexent- 0.0635 g/L |
| Pigments | 1. Leutine- 0.03975 g/L |
| | 2. Beta-Cryptoxanthin- 0.04521 g/L |
| | 3. Beta-Carotinal- 0.02912 g/L |
| Trace Elements | 1. Potassium-0.0700 g/L |
| | 2. Sodium- 0.0530 g/L |
| | 3. Calcium- 0.0230 g/L |
| | 4. Magnesium- 0.0070 g/L |
| | 5. Phosphorus- 0.0065 g/L |
| | 6. Iron- 0.0012 g/L |
| | 7. Zinc- 0.0007 g/L |
| | 8. Copper- 0.0005 g/L |
| | 9. Nickel- 0.0002 g/L |
| | 10. Chromium- 0.0003 g/L |
| Additives/ | 1. D-Glucose- 5.1001 g/L |
| Excipients | 2. 2-Phenyl-2-butenal- 0.0014 g/L |
| | 3. Sucrose- 0.0072 g/L |
| | 4. Chlorella Growth Factors- 0.0000005 g/L |
| | 5. Endotoxins- 0.0111 g/L |
| | 6. Phenol red- 0.0210 g/L |
| | 7. Sodium Pyruvate- 0.1211 g/L |
| | 8. HEPES (4-(2-hydroxyethyl)-1- |
| | piperazineethanesulfonic acid)- 6.0102 g/L |

23. A method for preparing the supplement composition as claimed in claim 21, comprising the steps of:
h. suspending components in water with continuous stirring to obtain a completely dissolved solution;
i. adjusting the pH of the solution of step (a) in the range of 7.2 to 7.5 by adding 1N acid or base drop by drop followed by adding water to adjust volume of the solution;
j. sterilizing the solution as obtained in step (b) by filtering through a sterile membrane filter under positive pressure to obtain a filtered solution;
k. aseptically adding growth factors and vitamins into the filtered solution obtained in step (c) to obtain the supplement composition; and
l. storing the supplement composition obtained from step (d) in a sterile container at a temperature of 2-8° C. in dark.

24. The method as claimed in claim 23, wherein the components used in step (a) are inorganic salts, amino acids, lipids, pigments, trace elements and additives/excipients.

25. The method as claimed in claim 23, wherein the acid/base in step (b) is selected from, but not limited to, 1N HCl and 1N NaOH.

26. The method as claimed in claim 23, wherein the pH of the solution is adjusted in the range of 7.0-7.5.

27. The method as claimed in claim 23, wherein the porosity of sterile membrane is in the range of 0.10 to 0.22 micron.

28. The method as claimed in claim 23, wherein the supplement composition is stored at a temperature 2-8° C. in dark environment.

29. The supplement composition as claimed in claim 21, wherein supplement composition supports the growth of cell lines.

30. The supplement composition as claimed in claim 29, wherein the cell lines are selected from, but not limited to, human breast cancer cell and human embryonic kidney cells and cervical cancer cell.

31. The supplement composition as claimed in claim 21, wherein viability of cells is in the range of 90-99% after 72 hours.
